(19) United States
(12) Patent Application Publication
(10) Pub. No.: US 2025/0108258 A1
(43) Pub. Date: Apr. 3, 2025

(54) Estimation of Motion Repetition using Local Magnetic Field Distortion

(71) Applicant: Apple Inc., Cupertino, CA (US)

(72) Inventors: Anselm Grundhoefer, Campbell, CA (US); Daniel Hellfeld, Lagune Niguel, CA (US); Hesam Najafi, Mountain View, CA (US); Manu Rastogi, San Jose, CA (US)

(21) Appl. No.: 18/817,782

(22) Filed: Aug. 28, 2024

Related U.S. Application Data
(60) Provisional application No. 63/586,708, filed on Sep. 29, 2023.

(51) Int. Cl. A63B 24/00; G01D 5/14; G16H 40/67

(52) U.S. Cl. CPC A63B 24/0062; G01D 5/14; G16H 40/67; A63B 2024/0071; A63B 2209/08; A63B 2220/17; A63B 2220/807; A63B 2220/836; A63B 2220/89; A63B 2230/62

(57) ABSTRACT

Predicting and counting repetitions of a physical activity includes capturing first sensor data, by a first magnetometer on a wearable device, a change in a magnetic field indicative of a ferromagnetic object moving in relation to the wearable device. One or more characteristics of a user motion are determined based on the first sensor data. A count of repetitions of the user motion are determined based on the one or more characteristics of the user motion, and a notification of the count of repetitions is generated.

### BACKGROUND

Current techniques in image data analysis provide for numerous insights into a scene depicted in an image. For example, object detection can be used to identify objects in a scene, or characteristics of an object in a scene. One application is to apply image data to a network to determine a pose of a person.

Shortfalls exist when it comes to predicting motion of an object. For example, in order to predict an activity undertaken by a person, a video sequence of frames may be fed into a network, and a prediction for the video sequence may be obtained based on the entirety of the video. Problems exist in obtaining real time predictions for a user activity without the use of image data.

### BRIEF DESCRIPTION OF THE DRAWINGS

FIGS. 1A-1B show example diagrams of a technique for estimating user activity, according to one or more embodiments.

FIG. 2 shows a flowchart of a technique for counting repetitions of predicted user activities, according to one or more embodiments.

FIG. 3 shows an example system diagram for using a headset to detect activity involving ferromagnetic objects, in accordance with one or more embodiments.

FIG. 4 shows, in flowchart form, a technique for determining user actions using multiple magnetometers, in accordance with one or more embodiments.

FIG. 5 shows, in flowchart form, a technique for determining motion characteristics of a user activity, according to one or more embodiments.

FIG. 6 shows an example system diagram of an electronic device, according to one or more embodiments.

FIG. 7 shows, in block diagram form, a simplified multifunctional device according to one or more embodiments.

### DETAILED DESCRIPTION

This disclosure is directed to systems, methods, and computer readable media for estimating motion based on local magnetic fields. In general, techniques described herein are directed to estimating user actions and repetitions using magnetometer data from a wearable device. In addition, techniques described herein are directed to managing repetition count for the estimated user actions.

According to one or more embodiments, a low power technique for estimating and tracking user activity is provided. In particular, a user can wear a device having a magnetometer and/or other sensors which can be used to detect change in a local magnetic field. The device can be worn while the user is performing a repetitive motion using one or more ferromagnetic objects. Based on the sensor data collected by the magnetometer, one or more characteristics of the user motion can be determined. For example, user action type, a rate of the motion, a number of repetitions, or the like can be determined. The count of repetitions can then be provided to a user via a notification.

In one or more embodiments, a wearable device may be situated with two or more magnetometers and/or other sensors which can be used to detect change in a local magnetic field. Sensor data can be captured by each of the magnetometers, and additional characteristics of the motion can be detected. For example, localization can be performed using the sensor data captured by each magnetometer, and the relative locations of the magnetometers on the head mounted device. For example, if a user is lifting a weight including ferromagnetic material, the combined sensor data can be used to determine the movement and position of the weight, and in turn, an estimated movement and position of an arm performing the action. By using magnetometer data, estimated user motion can be improved when the weight is outside a camera field of view.

In some embodiments, additional sensor data may be used to refine the estimation of the motion. For example, the head mounted device may additionally have a motion capturing sensor, such as an inertial measurement unit (IMU), from which an orientation of the device can be determined. An IMU refers to a system comprising an accelerometer and a gyroscope from which motion data can be determined. The sensor data can then be refined based on the orientation of the device to ensure accurate analysis of the sensor data even if a change in relative position of the magnetometers and ferromagnetic objects change due to a movement of the user.

According to one or more embodiments, the estimation of the user activity is performed by a low power device, and is designed to require minimal power or other resources. For example, the device may be a wearable device, such as a head mounted device, which is intended to be worn for long periods, and thus may be power constrained. Because the device may be power constrained, the device may rely on low-power sensors, such as magnetometers rather than cameras or other power-intensive sensors. Alternatively, the device can reduce the use of cameras or power-intensive sensors by shifting some of the sensor capture to lower powered sensors such as magnetometers, motion capture sensors, or the like.

In the following description, for purposes of explanation, numerous specific details are set forth in order to provide a thorough understanding of the disclosed concepts. As part of this description, some of this disclosure's drawings represent structures and devices in block diagram form in order to avoid obscuring the novel aspects of the disclosed embodiments. In this context, it should be understood that references to numbered drawing elements without associated identifiers (e.g., 100) refer to all instances of the drawing element with identifiers (e.g., 100a and 100b). Further, as part of this description, some of this disclosure's drawings may be provided in the form of a flow diagram. The boxes in any particular flow diagram may be presented in a particular order. However, it should be understood that the particular flow of any flow diagram is used only to exemplify one embodiment. In other embodiments, any of the various components depicted in the flow diagram may be deleted, or the components may be performed in a different order, or even concurrently. In addition, other embodiments may include additional steps not depicted as part of the flow diagram. The language used in this disclosure has been principally selected for readability and instructional purposes and may not have been selected to delineate or circumscribe the disclosed subject matter. Reference in this disclosure to “one embodiment” or to “an embodiment” means that a particular feature, structure, or characteristic described in connection with the embodiment is included in at least one embodiment, and multiple references to “one embodiment” or to “an embodiment” should not be understood as necessarily all referring to the same embodiment or to different embodiments.

It should be appreciated that in the development of any actual implementation (as in any development project), numerous decisions must be made to achieve the developers' specific goals (e.g., compliance with system and business-related constraints), and that these goals will vary from one implementation to another. It will also be appreciated that such development efforts might be complex and time consuming but would nevertheless be a routine undertaking for those of ordinary skill in the art of image capture having the benefit of this disclosure.

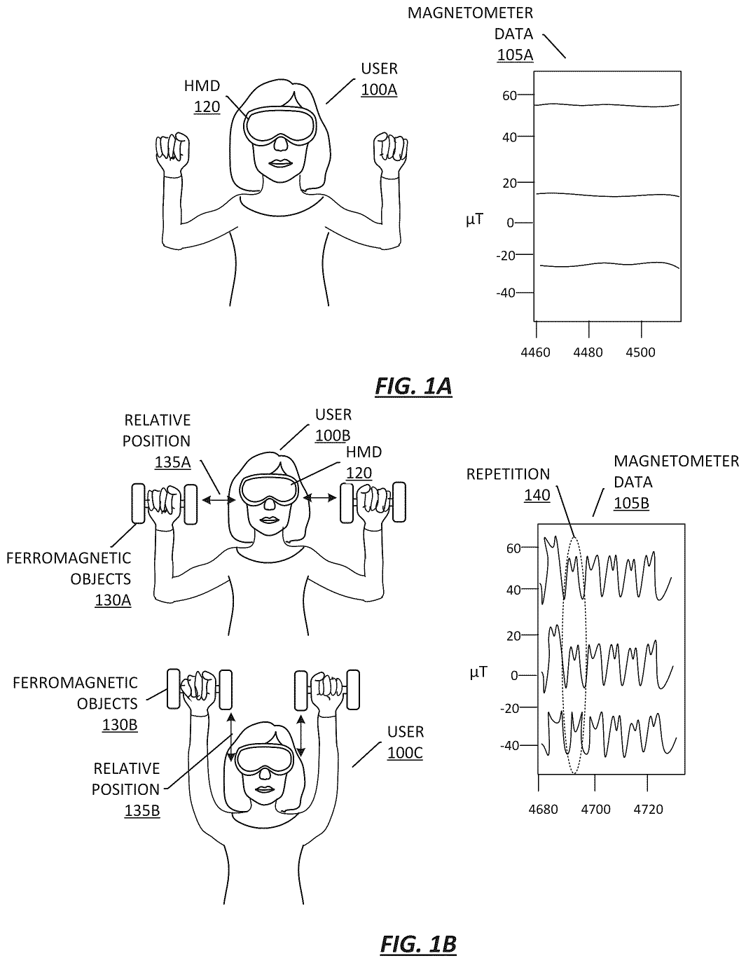

Referring to FIG. 1A, a diagram is presented in which a user is wearing a head mounted device 120. According to one or more embodiments, the head mounted device 120 may include sensors configured to collect data regarding the environment. In some embodiments, these sensors include one or more magnetometers. Each of the one or more magnetometers may include, for example, a 3-axis magnetometer configured to measure magnetic field displacement along an x, y, z coordinate system. In doing so, each of the magnetometers may be configured to detect the distortion of a local magnetic field as a ferromagnetic object moves in the vicinity.

According to some embodiments, the head mounted device 120 may include additional sensors which may be used to determine characteristics of the user motion. For example, the device may additionally include a motion detection sensor, such as an inertial measurement unit (IMU), gyroscope, accelerometer, or the like which is configured to measure the rotation and/or head-pose of the user. That is, because the head-mounted device is donned on a user's head, the orientation of the device as determined by the motion detection sensor data may be used to infer a pose of the user's head.

According to one or more embodiments, the one or more magnetometers may be continuously capturing magnetometer data 105A related to changes in the local magnetic field. From this data, the device 120 can estimate characteristics of the user motion with respect to ferromagnetic options. In FIG. 1A, the user 100A is not shown engaging with any ferromagnetic objects. As such, the magnetometer data 105A does not show any data related to changes in the local magnetic field tracked over the particular time period.

Turning to FIG. 1B, an example diagram is depicted of a user performing a motion using ferromagnetic objects 130 in the form of weights. For example, the weights may be comprised of iron or other ferromagnetic material. In a first position, the user 100B is shown in an initial pose of an overhead press motion. Accordingly, the ferromagnetic objects 130 are in a relative position 135A near the user's head and, thus, near the magnetometer in the HMD 120. According to one or more embodiments, the magnetometer in the HMD 120 may thus record a relatively strong distortion of the local magnetic field due to the proximity of the ferromagnetic objects 130 to the magnetometer in the HMD 120.

At a later time, as shown by the second position of user 100C, the user 100C is shown in a subsequent pose during the motion of an overhead press motion. Accordingly, the ferromagnetic objects 130 are in a relative position 135B and have moved away from the user's head and, thus, away from the magnetometer in the HMD 120. According to one or more embodiments, the magnetometer in the HMD 120 may thus record a reduced distortion of the local magnetic field due to the increased distance of the ferromagnetic objects 130 to the magnetometer in the HMD 120.

The resulting magnetometer data 105B shows the distortion of the local magnetic field over time during the motion. When the user 100 performs a repetitive motion using a ferromagnetic object that involves moving the ferromagnetic object to different positions in relation to the magnetometer, patterns of the relative positions may be tracked in the resulting magnetometer data. In some embodiments, one or more networks can be trained on different magnetometer data for different user motions and/or different ferromagnetic objects such that, during runtime, characteristics of the motion can be tracked. As an example, repetitive peaks in the magnetometer data 105B can indicate sets of repetitions. For example, repetition 140 is identified as a singular repetition of the overhead press performed by the user 100 because of the repeated pattern.

According to one or more embodiments, the HMD 120 may include additional sensor data which can be used to determine characteristics of the user motion. For example, the HMD 120 can include a motion capturing sensor, such as an IMU, gyroscope, accelerometer, or the like which capture sensor data related to the motion of the device from which an orientation of the device can be determined. In some embodiments, the orientation of the device as determined by the motion detection sensor data may be used to infer a pose of the user's head. The magnetometer data 105B can then be corrected to a static global frame. As another example, the HMD 120 may include one or more cameras, such as user-facing egocentric cameras, outward-facing scene cameras, or the like. In some embodiments, camera data captured by one or more of the cameras can capture image data from which user pose can be determined. In some embodiments, the HMD 120 may determine that the user has begun a motion to be tracked based on the camera data and may trigger the capture and/or analysis of the magnetometer data accordingly.

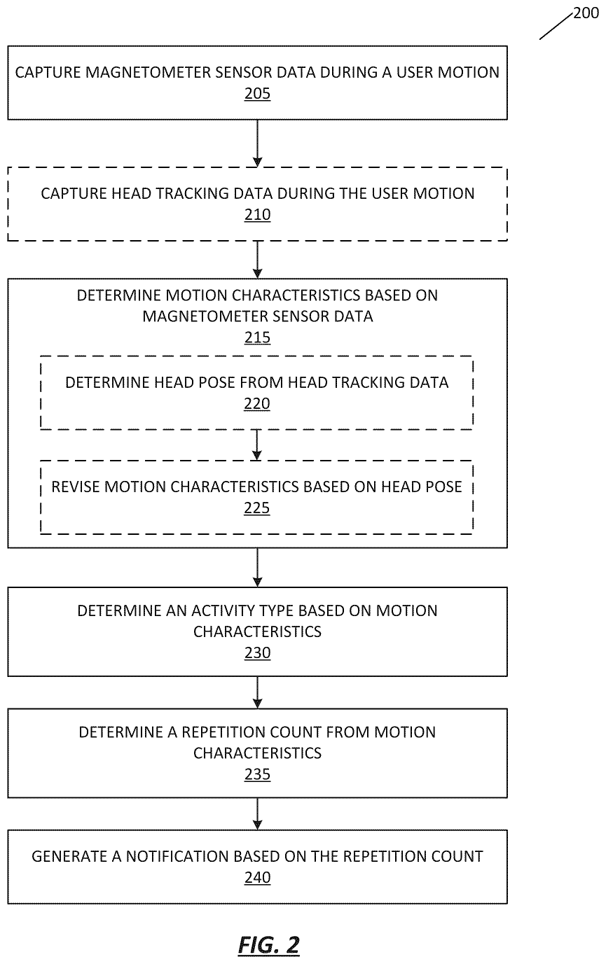

FIG. 2 shows a flowchart of a technique for counting repetitions of predicted user activities, according to one or more embodiments. For purposes of explanation, the following steps will be described in the context of particular components. However, it should be understood that the various actions may be taken by alternate components. As an example, a single system may perform all the actions described with respect to FIG. 2. Alternatively, separate components may perform the functions and the functionality may be distributed across multiple systems or devices. In addition, the various actions may be performed in a different order. Further, some actions may be performed simultaneously, and some may not be required, or others may be added.

The flowchart 200 begins at block 205, where magnetometer sensor data is captured during a user motion. According to one or more embodiments, the magnetometer may capture magnetic field displacement along an x, y, z coordinate system. In doing so, each of the magnetometers may be configured to detect the distortion of a local magnetic field as a ferromagnetic object moves in the vicinity. In one or more embodiments, the magnetometer may be integrated in a wearable device, such as a head mounted device, which is donned by a user while the user is performing the user motion.

At block 210, optionally, head tracking data is captured during the user motion. For example, the device may additionally include a motion detection sensor, such as an inertial measurement unit (IMU), gyroscope, accelerometer, or the like which is configured to measure the position and/or orientation of the device.

The flowchart 200 proceeds to block 215, where one or more motion characteristics of the user motion are determined based on the magnetometer sensor data. In particular, the motion characteristics may indicate a characteristic of how a user is causing a ferromagnetic object to move in relation to the magnetometer. According to one or more embodiments, the magnetometer sensor data may be applied to a network trained to detect patterns indicative of particular user motions. For example, a mode may be configured to classify the user motion based on the magnetometer sensor data. As described above with respect to FIG. 1B, a series of peaks may indicate a repeated movement of the ferromagnetic object in relation to the magnetometer in the device.

Optionally, a shown at block 220, head pose data may be determined from the head tracking data collected at block 210. According to one or more embodiments, the head pose data may be determined based on pose information for the device if the device is a head mounted device. Because the user motion is determined based on the relationship between the ferromagnetic object and the magnetometer, if a user moves their head while performing the user motion with the ferromagnetic object, the relative location between the ferromagnetic object and the head mounted device will include a component due to the user motion of the ferromagnetic object, and a component due to the head movement. Accordingly, at block 225, the motion characteristics can be determined, or revised, based on the head pose. As an example, the magnetometer data may be revised to a static global frame. For example, the data may be revised to reflect what the magnetic field displacement would have been if the head had stayed static during the user motion. As another example, a network trained to predict characteristics of the movement based on the magnetometer data may further be trained to consider head pose as an input into the network. That is, the network can be trained to distinguish head movements that correlate to an exercise or other activity, and head movements that are not related to the activity type. For example, rotation of the head, or greater translational movement of the head than is anticipated, such as if the user is performing a lunge movement.

At block 230, an activity type is determined based on the motion characteristics. In some embodiments, determining the activity type may include determining, based on the magnetometer sensor data, that a repetitive motion is being performed. Additionally, or alternatively, a class of activity may be determined based on the magnetometer data and/or other sensor data captured by the device. In some embodiments, the activity type is determined by a trained model configured to classify signatures in the magnetometer data and/or other sensor data base on activity type.

The flowchart 200 proceeds to block 235 where a repetition count for the activity is determined based on the motion characteristics. In some embodiments, the techniques described herein may be used by an application configured to track repetitive activity involving a ferromagnetic object, such as exercise involving lifting weights. For example, the network can be trained to detect repetitions of the signatures of the magnetometer data or other trends associated with the particular activity. In doing so, a repetition count may be determined based on the patterns detected in the magnetometer data. In some embodiments, the determination of the motion characteristics may be performed in real time, such that as the user performs additional repetitions, a repetition count can be updated.

The flowchart 200 concludes at block 235, where a notification is generated based on the repetition count. According to one or more embodiments, the repetition count can be reported to the user using visual feedback, audio feedback, or the like. For example, an audio announcement may be made for each repetition, or indicating a count of repetitions, by the device. As another example, the device may have a display, such as a pass-through display or a see-through display on which information regarding the repetition count is presented. In some embodiments, the notification may be transmitted to an additional device, such as a mobile device, accessory device, other wearable device, or the like. The additional device may then provide a notification to the user.

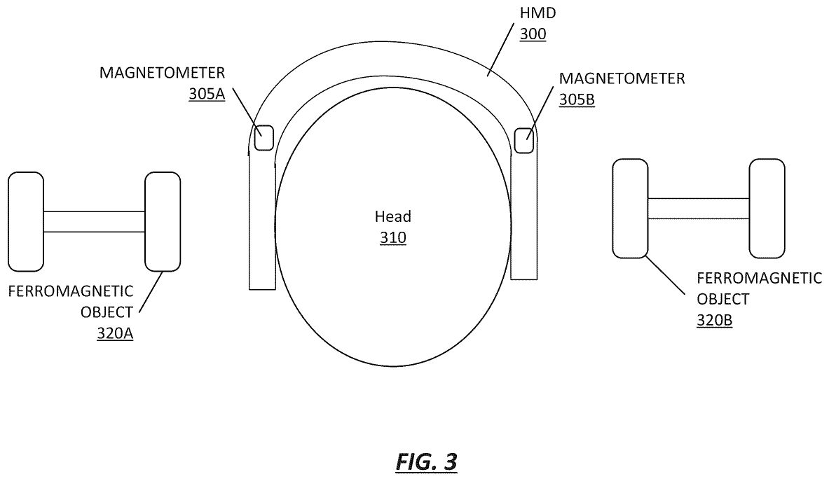

According to some embodiments, the wearable device may include multiple magnetometers. The sensor data captured by the set of magnetometers may provide additional data which can be used to determine additional characteristics of the user motion. FIG. 3 shows an example system diagram for using a headset to detect activity involving ferromagnetic objects, in accordance with one or more embodiments.

In the example diagram, an overhead view of a user using an HMD to track user motion involving ferromagnetic options. In particular, an HMD 300 is shown on the head 310 of a user. The HMD may include various sensors, including multiple magnetometers, as shown by magnetometer 305A and magnetometer 305B. According to one or more embodiments, the magnetometers may be situated in any part of the HMD 300. In some embodiments, the magnetometers may be integrated on opposite sides of the HMD 300 to enhance the field in which sensor data is obtained.

According to one or more embodiments, the use of multiple magnetometers may enhance localization techniques, and thereby allow the HMD 300 to determine additional characteristics about the user motion. As an example, as the user lifts ferromagnetic object 320A toward the head 310, both magnetometer 305A and magnetometer 305B may detect a corresponding change in local magnetic field. However, the change in local magnetic field will be stronger as detected by magnetometer 305A than the change in local magnetic field detected by magnetometer 305B at the same time. Based on a predefined spatial relationship between the magnetometer 305A and magnetometer 305B, and/or the known placement of magnetometer 305A and magnetometer 305B on HMD 300, location information for the ferromagnetic object 320 can be determined. For example, because the change in local magnetic field will be stronger as detected by magnetometer 305A than the change in local magnetic field detected by magnetometer 305B, and magnetometer 305A is known to be located on a left side of the device, whereas magnetometer 305B is known to be on a right side of the device, it can be determined that the movement of the ferromagnetic object 320A is performed by a left arm.

The use of two magnetometers also helps identify asynchronous motion of ferromagnetic objects. For example, if the user alternately raises ferromagnetic object 320A and ferromagnetic object 320B during an alternating bicep curl motion, then a pattern will emerge where at first magnetometer 305A detects a strong signal while magnetometer 305B detects a weak signal while ferromagnetic object 320A is raised. Then magnetometer 305B will detect a strong signal and magnetometer 305A will detect a weak signal when ferromagnetic object 320B is raised. Accordingly, not only can repetitions of the motion be counted, but characteristics of the alternating bicep curl can be detected by the collective sensor data.

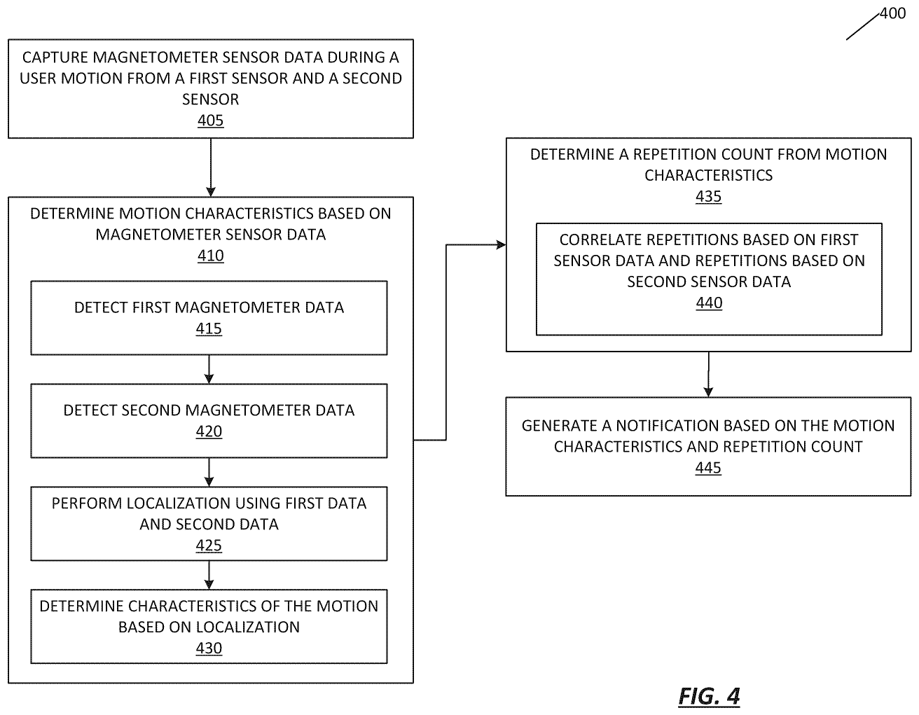

FIG. 4 shows, in flowchart form, a technique for determining user actions using multiple magnetometers, in accordance with one or more embodiments. For purposes of explanation, the following steps will be described in the context of particular components. However, it should be understood that the various actions may be taken by alternate components. As an example, a single system may perform all the actions described with respect to FIG. 2. Alternatively, separate components may perform the functions and the functionality may be distributed across multiple systems or devices. In addition, the various actions may be performed in a different order. Further, some actions may be performed simultaneously, and some may not be required, or others may be added.

The flowchart 400 begins at block 405, where magnetometer sensor data is captured during a user motion from multiple magnetometers including a first magnetometer and a second magnetometer. According to one or more embodiments, each magnetometer may capture magnetic field displacement along an x, y, z coordinate system associated with the particular magnetometer. In doing so, each of the magnetometers may be configured to detect the distortion of a local magnetic field as a ferromagnetic object moves in the vicinity. In one or more embodiments, the magnetometer may be situated in a wearable device, such as a head mounted device, in a manner such that the placement of the magnetometers within the wearable device and/or the relative location of the magnetometers may be known, for example, to a process used for determining characteristics of a user motion.

The flowchart continues to block 410, where one or more motion characteristics of the user motion are determined based on the magnetometer sensor data and/or other camera data or other sensor data. In particular, the motion characteristics may indicate a characteristic of how a user is causing a ferromagnetic object to move in relation to the magnetometers. According to one or more embodiments, the magnetometer sensor data may be applied to a network trained to detect patterns indicative of particular user motions. The flowchart 400 includes, at block 415, detecting first magnetometer data, and at block 420, detecting second magnetometer data. The first magnetometer data may be captured by a first magnetometer, while the second magnetometer data may be captured by a second magnetometer. In some embodiments, the sensor data from the first magnetometer may be applied to a network trained to detect patterns indicative of particular user motions separately from the second magnetometer. Further, a network may be trained to detect particular characteristics of a user motion based on input from multiple magnetometers on the device along with relational information for the magnetometers, such as the relative location of the magnetometers and/or a known location of the magnetometers on the device.

At block 425, localization is performed based on the sensor data from the first magnetometer and second magnetometer. According to one or more embodiments, performing localization using the sensor data from the first magnetometer and second magnetometer includes estimating a position and/or orientation of an object causing the change in the local magnetic field, such as the ferromagnetic object. As described above with respect to FIG. 3, one example of performing localization includes determining a relative location of the ferromagnetic object being handled by the user as compared to the HMD. In one or more embodiments, the sensor data may indicate a strength and direction of the magnetic field in the form of a vector in an x, y, z coordinate system. By comparing the vectors determined by both magnetometers, information related to the relative location of the ferromagnetic object in relation to the device can be determined.

The flowchart continues to block 430, where characteristics of the motion are determined based on the localization. For example, a determination may be made that a particular arm is performing the motion if the change in magnetic field is stronger on one side of the device than the other. As another example, a particular motion type can be determined based on the localization, such as a direction the user is moving the ferromagnetic object toward and away from the device.

At block 435, a repetition count is determined based on the motion characteristics. In some embodiments, the techniques described herein may be used by an application configured to track repetitive activity involving a ferromagnetic object, such as exercise involving lifting weights. In doing so, a repetition count may be determined based on the patterns detected in the magnetometer data. In some embodiments, the determination of the motion characteristics may be performed in real time, such that as the user performs additional repetitions, a repetition count can be updated.

In some embodiments, the sensor data from the first magnetometer and the second magnetometer can be used to determine a motion that includes two arms moving ferromagnetic objects. At block 440, the repetitions are correlated based on the first sensor data and second sensor data. Thus, in the example of the alternating bicep curls, a single magnetometer may detect a strong signal as the closer arm bring the weight toward the device, and a weaker signal as the further arm brings the weight toward the device. Rather than counting the two peaks as individual reps, by comparing the sensor data collected by each magnetometer, a determination can be made that the rep includes a motion from each arm. According to one or more embodiments, the magnetometer data may be used in combination to discern characteristics of the movement. In this example, the timing of the detected curls on each side can be compared to determine if the user is performing an alternating bicep curl or a synchronous bicep curl.

The flowchart 400 concludes at block 445, where a notification is generated based on the repetition count. According to one or more embodiments, the repetition count can be reported to the user using visual feedback, audio feedback, or the like. For example, an audio announcement may be made for each repetition, or indicating a count of repetitions, by the device. As another example, the device may have a display, such as a pass-through display or a see-through display on which information regarding the repetition count is presented. In some embodiments, the notification may be transmitted to an additional device, such as a mobile device, accessory device, other wearable device, or the like. The additional device may then provide a notification to the user.

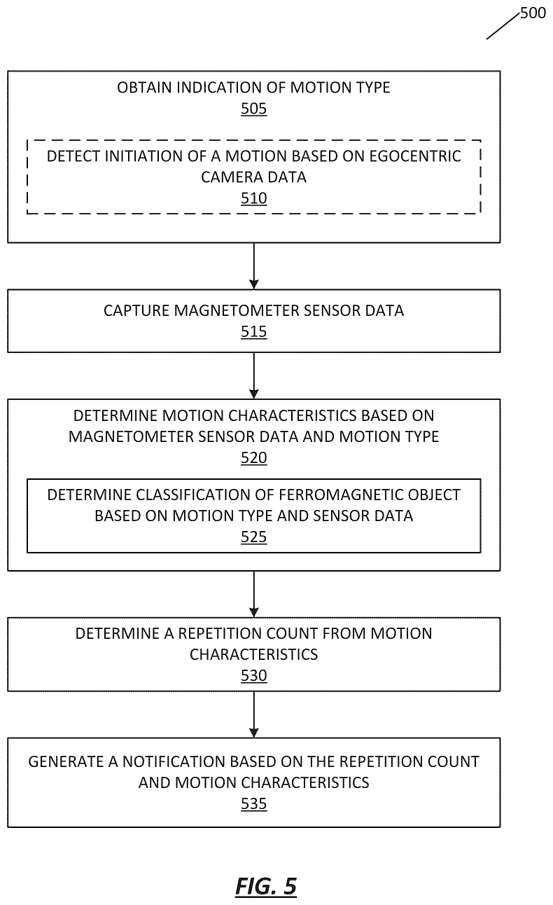

In some embodiments, the technique of estimating motion based on magnetometer data can be improved by collecting additional information about the movement. FIG. 5 shows, in flowchart form, a technique for determining motion characteristics of a user activity, according to one or more embodiments. For purposes of explanation, the following steps will be described in the context of particular components. However, it should be understood that the various actions may be taken by alternate components. As an example, a single system may perform all the actions described with respect to FIG. 2. Alternatively, separate components may perform the functions and the functionality may be distributed across multiple systems or devices. In addition, the various actions may be performed in a different order. Further, some actions may be performed simultaneously, and some may not be required, or others may be added.

The flowchart 500 begins at block 505, where an indication of the motion type is obtained. According to one or more embodiments, the indication can be detected by one or more sensors different than the magnetometers. For example, as shown in optional block 510, an indication of the motion can be detected based on egocentric camera data. That is, a head mounted device can capture image data of a user, and use body tracking or other techniques to estimate a pose or action being performed by the user. According to one or more embodiments, if the body tracking or other tracking data indicates that the user is performing a motion to be tracked by magnetometer data, then the estimation process can be initiated. In some embodiments, the egocentric cameras can then switch to a low power mode or are powered down while the activity is being performed by the user and repetitions are tracked using the magnetometer data, thereby conserving resources on the head mounted device. In this scenario, the cameras may be powered on and use for determining a motion type if the magnetometer sensor data no longer corresponds to the determined motion type. In some embodiments, the magnetometer may be used to track the user motion when the weights are out of the field of view of the cameras. As another example, obtaining an indication of the motion type may include receiving a notification from user input, a remote device, or the like indicating that a user is performing a particular repetitive motion.

Additionally, or alternatively, other sensor data may be captured for obtaining an indication of a motion type. For example, the device may additionally include an IMU which collects motion data for the device. In some embodiments, the IMU data may be considered for determination of motion type. For example, the motion type may be determined based on a determination that a first portion of the user motion is attributable to the movement of the ferromagnetic object (for example, captured by the magnetometer) and a second portion of the user motion is attributable to a movement of a body of the user (for example, captured by the IMU). As an example, while a user is performing a lunge while holding weights, an IMU may indicate a translational movement of the user, but the magnetometer may indicate a smaller amount of movement as the user is holding the weights to their side. Thus, the magnetometer data alone may not be indicative of the motion type, while the magnetometer data along with the IMU data provides more context for the motion type.

The flowchart 500 continues to block 515, where magnetometer sensor data is captured by one or more magnetometers. In one or more embodiments, the one or more magnetometers are triggered in response to the estimation process beginning, as described above with respect to block 505. Alternatively, the magnetometer may be continuously capturing data related to a local magnetic field, and the resulting sensor data can be analyzed to estimate motion characteristics in response to the received indication of the motion type. For example, captured magnetometer data may be compared with previously captured magnetometer data which was captured along with camera data from which computer vision can be performed. The previously captured magnetometer data can then be used as a reference for the currently captured magnetometer data to determine whether a same exercise is being performed as is detected by computer vision techniques in the camera data.

At block 520, motion characteristics are determined based on the magnetometer sensor data and/or motion type. For example, if the device receives an indication of a motion type being performed, a network used to estimate the motion can further refine the estimation. For example, magnetometer data associated with a bicep curl may have certain characteristics, either for the specific exercise, or for a particular user performing the exercise. As such, if a known user is performing a known exercise, the resulting sensor data can be compared against expected data to determine characteristics of the motion. As an example, a user may perform an overhead press the same way such that a particular pattern in the sensor data can be expected for a given weight. Accordingly, if the pattern differs, then additional data can be learned about the motion, such as a weight or weight class of the ferromagnetic object handled by the user performing the motion. That is, a heavier weight will result in a stronger signal than a lighter weight when the user is performing the same motion with different weights. Accordingly, a weight or weight class of the ferromagnetic object can be determined, in accordance with one or more embodiments. Thus, a classification of the ferromagnetic object can be determined, in accordance with one or more embodiments.

The flowchart 500 proceeds to block 530 where a repetition count for the motion is determined based on the motion characteristics. In some embodiments, the techniques described herein may be used by an application configured to track repetitive activity involving a ferromagnetic object, such as exercise involving lifting weights. In doing so, a repetition count may be determined based on the patterns detected in the magnetometer data. In some embodiments, the determination of the motion characteristics may be performed in real time, such that as the user performs additional repetitions, a repetition count can be updated.

The flowchart 500 concludes at block 535, where a notification is generated based on the repetition count. According to one or more embodiments, the repetition count can be reported to the user using visual feedback, audio feedback, or the like. For example, an audio announcement may be made for each repetition, or indicating a count of repetitions, by the device. As another example, the device may have a display, such as a pass-through display or a see-through display on which information regarding the repetition count is presented. In some embodiments, the notification may be transmitted to an additional device, such as a mobile device, accessory device, other wearable device, or the like. The additional device may then provide a notification to the user.

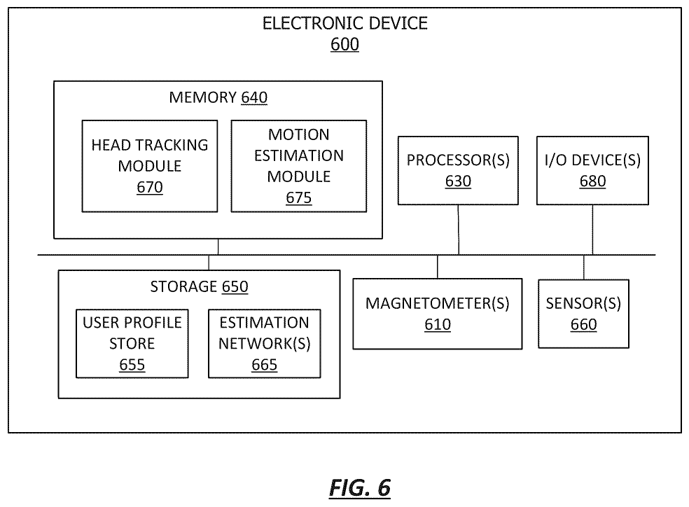

Referring to FIG. 6, a simplified block diagram of an electronic device 600 is depicted, in accordance with one or more embodiments of the disclosure. Electronic device 600 may be part of a multifunctional device, such as a mobile phone, tablet computer, personal digital assistant, portable music/video player, wearable device, or any other electronic device that includes a camera system. FIG. 6 shows, in block diagram form, an overall view of a system diagram capable of supporting proximity detection and breakthrough, according to one or more embodiments. Electronic device 600 may be connected to other network devices across a network via network interface, such as mobile devices, tablet devices, desktop devices, as well as network storage devices such as servers and the like. In some embodiments, electronic device 600 may communicably connect to other electronic devices via local networks to share sensor data and other information.

Electronic Device 600 may include one or more processors 630, such as a central processing unit (CPU). Processor 630 may be a system-on-chip such as those found in mobile devices and include one or more dedicated graphics processing units (GPUs). Further, processor 630 may include multiple processors of the same or different type. Electronic Device 600 may also include a memory 640. Memory 640 may include one or more different types of memory, which may be used for performing device functions in conjunction with processor 630. For example, memory 640 may include cache, ROM, and/or RAM. Memory 640 may store various programming modules during execution, including head tracking module 670, and motion estimation module 675. According to some embodiments, motion estimation module 675 may provide a user with activity-based tracking and feedback based on motion characteristics determined from sensor data, such as magnetometer(s) 610. As an example, motion estimation module 675 may include health applications, exercise applications, or other application where predicting and tracking user activity is utilized. Head tracking module 670 may utilize data from head tracking sensor data from one or more sensor(s) 660, such as motion tracking sensors, from which a head pose or position can be derived. Motion estimation module 675 may include functionality for utilizing the head tracking data to refine an estimated activity being performed by a person. Motion estimation module may utilize a network trained to generate predictions for characteristics of one or more activities based on magnetometer data and/or other sensor data from other sensor 660, such as cameras, motion tracking sensors, or the like. The electronic device may include one or more storage devices 650, which may be used to hold data to facilitate processing of head tracking module 670, and/or motion estimation module 675. In particular, storage 650 may include user profile store 655 which may include user specific information, such as predefined motions, user data, or the like. Storage 650 may also include Estimation network(s) 665. Estimation network(s) 665 may include one or more networks trained to determine characteristic of a motion and count repetitions of a motion based on sensor data, for example from magnetometer 610, and/or other sensors 660.

Sensors 660 may include one or more cameras. The cameras may each include an image sensor, a lens stack, and other components that may be used to capture images. In one or more embodiments, the cameras may be directed in different directions in the electronic device. For example, a front-facing camera may be positioned in or on a first surface of the electronic device 600, while the back-facing camera may be positioned in or on a second surface of the electronic device 600. In some embodiments, the cameras may include one or more types of cameras, such as RGB cameras, depth cameras, and the like. One or more of the cameras may include egocentric cameras, which are configured to capture image data comprising the user.

In one or more embodiments, the electronic device 600 may also include a display 680. Display 680 may be any kind of display device, such as an LCD (liquid crystal display), LED (light-emitting diode) display, OLED (organic light-emitting diode) display, or the like. In addition, display 680 could be a semi-opaque display, such as a heads-up display, pass-through display, or the like. Rather than an opaque display, a head mountable system may have a transparent or translucent display. The transparent or translucent display may have a medium through which light representative of images is directed to a person's eyes. The display may utilize digital light projection, OLEDs, LEDs, uLEDs, liquid crystal on silicon, laser scanning light source, or any combination of these technologies. The medium may be an optical waveguide, a hologram medium, an optical combiner, an optical reflector, or any combination thereof. In some implementations, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface.

Although electronic device 600 is depicted as comprising the numerous components described above, in one or more embodiments, the various components may be distributed across multiple devices. Further, additional components may be used and/or some combination of the functionality of any of the components may be combined.

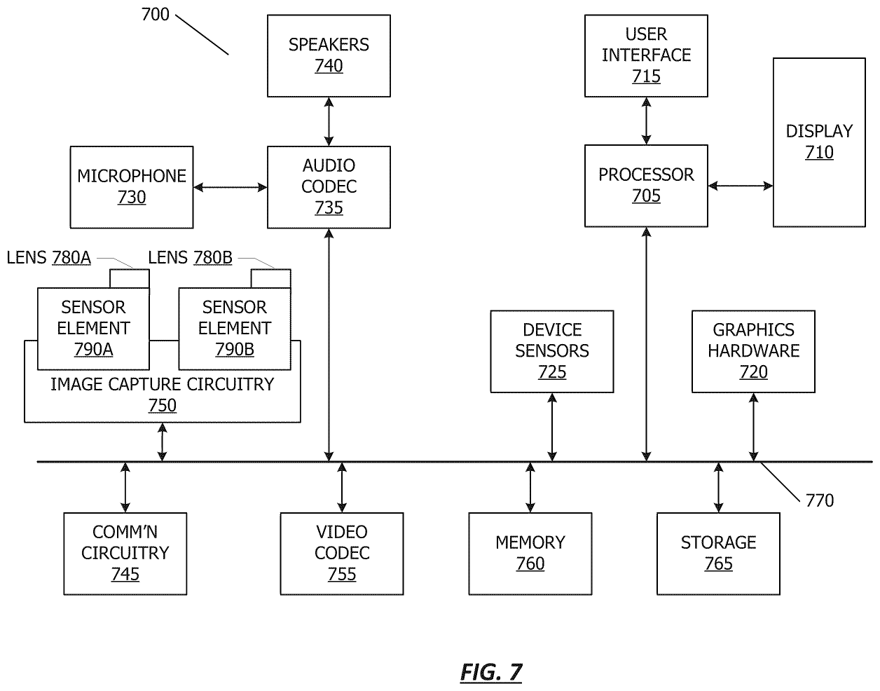

Referring now to FIG. 7, a simplified functional block diagram of illustrative multifunction device 700 is shown according to one embodiment. Multifunction electronic device 700 may include processor 705, display 710, user interface 715, graphics hardware 720, sensors 725 (e.g., proximity sensor/ambient light sensor, accelerometer and/or gyroscope), microphone 730, audio codec(s) 735, speaker(s) 740, communications circuitry 745, digital image capture circuitry 750 (e.g., including camera system), video codec(s) 755 (e.g., in support of digital image capture unit), memory 760, storage device 765, and communications bus 770. Multifunction electronic device 700 may be, for example, a digital camera or a personal electronic device such as a personal media player, mobile telephone, head-mounted device, or a tablet computer.

Processor 705 may execute instructions necessary to carry out or control the operation of many functions performed by device 700 (e.g., the generation and/or processing of images as disclosed herein). Processor 705 may, for instance, drive display 710 and receive user input from user interface 715. User interface 715 may allow a user to interact with device 700. For example, user interface 715 can take a variety of forms, such as a button, keypad, dial, a click wheel, keyboard, display screen and/or a touch screen. Processor 705 may also, for example, be a system-on-chip such as those found in mobile devices and include a dedicated graphics processing unit (GPU). Processor 705 may be based on reduced instruction-set computer (RISC) or complex instruction-set computer (CISC) architectures or any other suitable architecture and may include one or more processing cores. Graphics hardware 720 may be special purpose computational hardware for processing graphics and/or assisting processor 705 to process graphics information. In one embodiment, graphics hardware 720 may include a programmable GPU.

Image capture circuitry 750 may include two (or more) lens assemblies 780A and 780B, where each lens assembly may have a separate focal length. For example, lens assembly 780A may have a short focal length relative to the focal length of lens assembly 780B. Each lens assembly may have a separate associated sensor element 790. Alternatively, two or more lens assemblies may share a common sensor element. Image capture circuitry 750 may capture still and/or video images. Output from image capture circuitry 750 may be processed, at least in part, by video codec(s) 755, and/or processor 705, and/or graphics hardware 720, and/or a dedicated image processing unit or pipeline incorporated within circuitry 750. Images so captured may be stored in memory 760 and/or storage 765.

Sensor and camera circuitry 750 may capture still and video images that may be processed in accordance with this disclosure, at least in part, by video codec(s) 755, and/or processor 705, and/or graphics hardware 720, and/or a dedicated image processing unit incorporated within circuitry 750. Images so captured may be stored in memory 760 and/or storage 765. Memory 760 may include one or more different types of media used by processor 705 and graphics hardware 720 to perform device functions. For example, memory 760 may include memory cache, read-only memory (ROM), and/or random-access memory (RAM). Storage 765 may store media (e.g., audio, image, and video files), computer program instructions or software, preference information, device profile information, and any other suitable data. Storage 765 may include one more non-transitory computer-readable storage mediums including, for example, magnetic disks (fixed, floppy, and removable) and tape, optical media such as CD-ROMs and digital video disks (DVDs), and semiconductor memory devices such as Electrically Programmable Read-Only Memory (EPROM), and Electrically Erasable Programmable Read-Only Memory (EEPROM). Memory 760 and storage 765 may be used to tangibly retain computer program instructions or code organized into one or more modules and written in any desired computer programming language. When executed by, for example, processor 705, such computer program code may implement one or more of the methods described herein.

It is to be understood that the above description is intended to be illustrative and not restrictive. The material has been presented to enable any person skilled in the art to make and use the disclosed subject matter as claimed and is provided in the context of particular embodiments, variations of which will be readily apparent to those skilled in the art (e.g., some of the disclosed embodiments may be used in combination with each other). Accordingly, the specific arrangement of steps or actions shown in FIGS. 2 and 4-5, or the arrangement of elements shown in FIGS. 1, 3, and 6-7, should not be construed as limiting the scope of the disclosed subject matter. The scope of the disclosed subject matter should be determined with reference to the appended claims, along with the full scope of equivalents to which such claims are entitled. In the appended claims, the terms “including” and “in which” are used as the plain-English equivalents of the respective terms “comprising” and “wherein.”

1. A method comprising:
capturing first sensor data, by a first magnetometer on a wearable device, a change in a magnetic field indicative of a ferromagnetic object moving in relation to the wearable device;
determining, based on the first sensor data, one or more characteristics of a user motion;
determining a count of repetitions of the user motion based on the one or more characteristics of the user motion; and
generating a notification of the count of repetitions.

2. The method of claim 1, wherein determining the one or more characteristics comprises:
applying the first sensor data to a model configured to classify user motion based on sensor data from the first magnetometer.

3. The method of claim 1, further comprising:
capturing second sensor data by a second magnetometer, wherein determining the one or more characteristics of the user motion is further based on the second sensor data.

4. The method of claim 3, wherein the wearable device is a head mounted device, and wherein the first magnetometer is integrated into a first side of the head mounted device, and the second magnetometer is situated into a second side of the head mounted device such that the first magnetometer and the second magnetometer are located on different sides of a user when the head mounted device is donned by a user.

5. The method of claim 3, wherein the one or more characteristics of the user motion comprises determining an arm performing the user motion.

6. The method of claim 1, further comprising:
capturing head tracking sensor data from one or more additional sensors; and
determining a head position based on the head tracking sensor data,
wherein one or more characteristics of the user motion are further determined based on the head position.

7. The method of claim 1, further comprising:
detecting an initiation of a motion type from one or more additional sensors; and
wherein determining the one or more characteristics of the user motion is triggered in response to the detected initiation of the motion type.

8. The method of claim 1, further comprising:
capturing second sensor data by one or more cameras;
determining a classification of the user motion based on the first sensor data and the second sensor data; and
in response to determining the classification:
powering down the one or more cameras, and
determining the count of repetitions based on additional sensor data captured by the first magnetometer and not the one or more cameras.

9. A non-transitory computer readable medium comprising computer readable code executable by one or more processors to:
capture first sensor data, by a first magnetometer on a wearable device, a change in a magnetic field indicative of a ferromagnetic object moving in relation to the wearable device;
determine, based on the first sensor data, one or more characteristics of a user motion;
determine a count of repetitions of the user motion based on the one or more characteristics of the user motion; and
generate a notification of the count of repetitions.

10. The non-transitory computer readable medium of claim 9, wherein the computer readable code to determine the one or more characteristics comprises computer readable code to:
apply the first sensor data to a model configured to classify user motion based on sensor data from the first magnetometer.

11. The non-transitory computer readable medium of claim 9, further comprising computer readable code to:
capture second sensor data by a second magnetometer, wherein determining the one or more characteristics of the user motion is further based on the second sensor data.

12. The non-transitory computer readable medium of claim 11, wherein the wearable device is a head mounted device, and wherein the first magnetometer is integrated into a first side of the head mounted device, and the second magnetometer is situated into a second side of the head mounted device such that the first magnetometer and the second magnetometer are located on different sides of a user when the head mounted device is donned by a user.

13. The non-transitory computer readable medium of claim 11, wherein the one or more characteristics of the user motion comprises determining an arm performing the user motion.

14. The non-transitory computer readable medium of claim 9, further comprising computer readable code to:
capture head tracking sensor data from one or more additional sensors; and
determine a head position based on the head tracking sensor data,
wherein one or more characteristics of the user motion are further determined based on the head position.

15. The non-transitory computer readable medium of claim 9, further comprising computer readable code to:
detect an initiation of a motion type from one or more additional sensors; and
wherein determining the one or more characteristics of the user motion is triggered in response to the detected initiation of the motion type.

16. The non-transitory computer readable medium of claim 9, further comprising computer readable code to:
capture second sensor data by an inertial measurement unit; and
determine a classification of the user motion based on the first sensor data and the second sensor data,
wherein the classification is based on a determination that a first portion of the user motion is attributable to a movement of the ferromagnetic object and a second portion of the user motion is attributable to a movement of a body of the user.

17. A system comprising:
one or more processors; and
one or more computer readable media comprising computer readable code executable by the one or more processors to:
capture first sensor data, by a first magnetometer on a wearable device, a change in a magnetic field indicative of a ferromagnetic object moving in relation to the wearable device;
determine, based on the first sensor data, one or more characteristics of a user motion;
determine a count of repetitions of the user motion based on the one or more characteristics of the user motion; and
generate a notification of the count of repetitions.

18. The system of claim 17, wherein the computer readable code to determine the one or more characteristics comprises computer readable code to:
apply the first sensor data to a model configured to classify user motion based on sensor data from the first magnetometer.

19. The system of claim 17, further comprising computer readable code to:
capture second sensor data by a second magnetometer, wherein determining the one or more characteristics of the user motion is further based on the second sensor data.

20. The system of claim 17, further comprising computer readable code to:
capture second sensor data by one or more cameras;
determine a classification of the user motion based on the first sensor data and the second sensor data; and
in response to determining the classification:
power down the one or more cameras, and
determine the count of repetitions based on additional sensor data captured by the first magnetometer and not the one or more cameras.
